(19) United States
(12) Patent Application Publication
(10) Pub. No.: US 2009/0075963 A1
(43) Pub. Date: Mar. 19, 2009

(54) TRANSDERMAL HORMONE SPRAY

(75) Inventors: R. Saul Levinson, Chesterfield, MO (US); Larry G. Miller, Saint Charles, MO (US)

Correspondence Address: KV PHARMACEUTICAL COMPANY, 4080B WEDGEWAY COURT, EARTH CITY, MO, 63045, US

(73) Assignee: DRUGTECH CORPORATION, Wilimington, DE (US)

(21) Appl. No.: 12/209,961

(22) Filed: Sep. 12, 2008

Related U.S. Application Data
(60) Provisional application No. 60/993,755, filed on Sep. 14, 2007.

(51) Int. Cl. A61K 31/565

(52) U.S. Cl. 514/182

(57) ABSTRACT

The present invention provides a transdermal spray drug delivery system which comprises: a therapeutically effective amount of a hormone; at least one dermal penetration enhancer; and at least one volatile liquid. The invention also provides a method for administering at least one systemic acting hormone which comprises applying an effective amount of the hormone in the form of the drug delivery system of the present invention.

This application claims the benefit of U.S. provisional patent application Ser. No. 60/993,755 filed Sep. 14, 2007, the entire disclosure of which is incorporated herein by reference.

### FIELD OF THE INVENTION

The present invention relates to hormone compositions administered to the skin via a spray or mist and to hormone delivery systems for the transdermal delivery of a metered quantity of the hormone composition to the skin via a spray or mist. The present invention also relates to methods for the treatment of conditions associated with low hormone levels in a patient by the application to the skin of a spray or mist comprising the hormone composition of the present invention. Transdermal hormone compositions and methods for their delivery of the present invention may be used for systemic delivery of the hormone.

### BACKGROUND OF THE INVENTION

There is a need for methods for the safe and effective administration of physiologically active agents, such as hormones. For many medications it is important that the administration regime be as simple and non-invasive as possible in order to maintain a high level of compliance by a patient. Oral administration is one administration regime that is commonly used because it is a relatively simple regime. However, the oral administration route is associated with complications including gastrointestinal irritation, highly variable blood levels, and drug metabolism in the liver.

Administration of physiologically active agents through the skin (‘transdermal drug delivery’) has received increased attention because it not only provides a relatively simple dosage regime, but it also provides a relatively slow and controlled route for release of a physiologically active agent into the systemic circulation. However, transdermal drug delivery is complicated by the fact that the skin behaves as a natural barrier to the agent entering systemic circulation.

Structurally, the skin consists of two principle parts, a relatively thin outermost layer (the epidermis) and a thicker inner region (the dermis). The outermost layer of the epidermis (the stratum corneum) consists of flattened dead cells, which are filled with keratin. Between the flattened dead cells of the stratum corneum are lipids, which form lamellar phases that are responsible for the natural barrier properties of the skin.

For effective transdermal delivery of a physiologically active agent that is applied to the surface of the skin (topical application), the agent must be partitioned from the vehicle into the stratum corneum, it must typically then be diffused within the stratum corneum before being partitioned from the stratum corneum to the viable epidermis and then into the dermal circulation.

To overcome some of the problems with transdermal delivery that are associated with transport across the dermal layers (percutaneous absorption), physiologically active agents are commonly formulated with one or more dermal penetration enhancers (Finnin and Morgan, J. Pharm. Sci., Vol. 88, No. 10, October 1999, pp 955-958), which are often lipophilic chemicals that readily partition into the stratum corneum whereupon they exert their effects on improving the transport of drugs across the skin barrier.

Many transdermal drug delivery systems have employed patches that are adhered to the skin for hours or days to deliver the drug across the skin and into the bloodstream at effective concentrations. However, transdermal patches suffer from a number of drawbacks, including skin irritation; poor cosmetic acceptability; expensive manufacturing processes; and limited flexibility for adjusting the dose. Complex manufacturing steps that may be required with transdermal patches include, for example, thermal blending of adhesive; precision coating onto the release liner; controlled drying of the patch; laminating of the backing of the patch; and packaging in a foil-lined pack. (Jenkins A. W., Developing the FEMATRIX™ transdermal patch, Pharm. J. 1995, Vol. 255, August 5 pp. 179-181).

A number of drawbacks associated with patches can sometimes be avoided with conventional topical vehicles, such as gels, creams and lotions. However, the use of these vehicles for transdermal drug delivery has been constrained by their limited application to the full range of transdermal drug candidates due to a low transdermal flux. Gels, creams and lotions may also suffer from messy application methods; poor dosage control during application; unacceptably long drying-times on the skin; and the potential for significant transfer of the drug from the patient to a caregiver or partner.

The foregoing problems have led to the development of devices for the controlled application of volatile:nonvolatile liquid formulations to the skin, such as that disclosed in U.S. Pat. No. 6,113,008, which describes a device for applying an occlusive spray-on bandage to the skin. However, that device suffers a number of limitations including no means to prevent actuator nozzle blockage by the preferred film-forming aerosols during its normal use, and restriction by its axial nozzle orientation to practical application to the forearm only, unless the patient is willing to lie down during operation of the device for application to other traditional sites such as the abdomen, upper buttocks and thigh. Such an approach would then also pose the additional problem of a lack of dip-tube pick-up from the liquid reservoir. Solutions, such as that described in U.S. Pat. No. 5,624,060, would add significant cost and complexity to the utility of the device disclosed by U.S. Pat. No. 6,113,008. That device also relies upon a vent in the dispensing shroud that is open to ambient air in order to prevent any pressure build-up when using the preferred pressurized aerosols, but leads to spray drift with consequent loss of some of the dispensed substance.

U.S. Pat. No. 6,261,274 discloses another dispensing device that utilizes a distance-gauging means to control the distance and orientation of the actuator nozzle from the skin. That device also suffers from limitations including the likelihood of variable actuator nozzle angle and/or distance relative to the skin, notwithstanding the use of a flat surface at the end of the distance-gauging means that is pressed against the skin during use. That is because in practice, the surface area of this feature needs to be restricted in order to avoid encroachment upon the dispensed substance plume during normal use of the device. The typical length required for the distance-gauge would mean that little if any stabilizing effect is achieved during normal use, because the angle of the distance-gauge would be prone to angle changes due to normal compliance of the skin surface, a problem which is compounded by only having limited surface area of contact for the distance-gauge, as well as the leveraged effect that even a small change in the angle of the distance-gauge has on the distance and angle of the actuator nozzle relative to the skin. The device of U.S. Pat. No. 6,261,274 also suffers from the limitation that the patient needs to be able to assess the actual site at which the dispensed substance would be applied, which is a particular problem for application to the forearm, where the spacer leg could be placed on the skin but the substance then sprayed into the air, missing the skin altogether. The device therefore suffers from a high potential for variation in the surface area over which the dispensed substance is applied, and the possibility to miss the target application site.

Substance dispensing devices of the foregoing kind tend to suffer an unacceptable loss of the substance in the period between uses of the device. That loss is particularly evident in circumstances involving use of a volatile substance or solvent. Unintentional loss of the substance is wasteful, and can also interfere with the ability of the device to dispense an accurately metered quantity each time the device is operated. In that regard, accurate metering can be very important in some circumstances.

### BRIEF SUMMARY OF THE INVENTION

The present invention is directed to a transdermal spray for delivering systemically a therapeutically effective amount of a hormone. In one embodiment, the invention provides a transdermal estrogen spray composition, comprising a therapeutically effective amount of an estrogen, a penetration enhancer, and a volatile solvent.

In one embodiment, the penetration enhancer is selected from terpenes, terpenoids, essential oils, pyrrolidones, 1-alkylazacycloheptan-2-ones, fatty acids, fatty acid esters, sulfoxides, amides, alcohols, glycols and glycerides, amino acid derivatives, phospholipids, and surfactants.

In a further embodiment, the penetration enhancer has the formula Iₐ or I_{b}:

R¹—C(═O)—O—H Iₐ

or

R²—C(═O)—O—R³ I_{b}

wherein:
R¹ is selected from a straight chain, branched, or cyclic-containing alkyl group or substituted alkyl group having 8 to 20 carbons, and a straight chain, branched, or cyclic-containing alkenyl group or substituted alkenyl group having 8 to 20 carbons;
R² is selected from a straight chain, branched, or cyclic-containing alkyl group or substituted alkyl group having 8 to 20 carbons, and a straight chain, branched, or cyclic-containing alkenyl group or substituted alkenyl group having 8 to 20 carbons; and
R³ is selected from lower alkyl, lower alkenyl, a straight chain, branched, or cyclic-containing alkyl group or substituted alkyl group having 6 to 14 carbons, and a straight chain, branched, or cyclic-containing alkenyl group or substituted alkenyl group having 6 to 14 carbons; and
the substituted alkyls or substituted alkenyls have from 1 to 4 substituents selected from hydroxy, halo, oxo, alkoxy, and amino.

In another embodiment, the penetration enhancer is selected from the group consisting of butylated hydroxyanisole, 2-phenoxyethanol, thymol, menthol, menthone, cineole, isopropyl myristate, glyceryl monolaurate, glyceryl monostearate, glyceryl monooleate, oleic acid, oleyl alcohol, methyl laurate, sorbitan monooleate, lauryl lactate, and lauryl alcohol.

In one embodiment, when applied to the skin, the composition provides an in vivo flux rate of about 0.03 to about 0.3 μg/cm² per hour.

In one embodiment, the hormone in the transdermal spray is an estrogen.

In another embodiment, the estrogen is estradiol.

In one embodiment, the solvent in the transdermal hormone spray is selected from ethanol and isopropanol.

### DETAILED DESCRIPTION OF THE INVENTION

The present invention provides a transdermal drug delivery system comprising: (a) a therapeutically effective amount of one or more hormones; (b) at least one dermal penetration enhancer; and (c) at least one volatile liquid incorporated into a dosage form for topical application and delivered to the skin by a device capable of delivering a metered quantity of a hormone composition via a spray or mist. The hormone delivery system of the present invention enables a wide range of hormones to be delivered through the skin to achieve a desired systemic effect. The hormone delivery system preferably comprises a hormone composition in which the hormone is intimately mixed with a dermal penetration enhancer and a volatile liquid. Where the hormone composition is applied to the skin, the hormone and penetration enhancer are thermodynamically driven into the skin as the volatile liquid evaporates. Once within the skin the penetration enhancer may either disrupt the lipid matrix and/or act as a solubilizer to allow an enhanced penetration rate of the hormone through the skin and into the circulation. In this way, the dermal penetration enhancer acts as a vehicle for the hormone, aiding in the penetration of the hormone into the skin.

Once the volatile liquid of the drug delivery system has evaporated, driving the mixture of dermal penetration enhancer and hormone into the stratum corneum, the outer surface of the skin is then substantially free of hormone and dermal penetration enhancer. Normal touching, wearing of clothes, rinsing or even washing of the skin will not, to any significant extent, affect delivery of the hormone or displace either the hormone or the dermal penetration enhancer, once the volatile liquid has evaporated.

The rate of absorption of the hormone through the stratum corneum is increased by the dermal penetration enhancer. The hormone is solublized by the dermal penetration enhancer at the time when it is being transported from the surface of the skin and into the stratum corneum. The performance of the dermal penetration enhancer in delivering a desired hormone varies with differences in both the nature of the dermal penetration enhancer and the hormone.

It is believed that the dermal penetration enhancer is readily absorbed into the stratum corneum in sufficient quantities to form a reservoir or depot of the dermal penetration enhancer within the stratum corneum. As the dermal penetration enhancer crosses through the skin to form the skin-depot, the hormone contained therein is transported through the skin and is contained within the depot. These depots are believed to form within the lipid matrix of the stratum corneum, wherein the lipid matrix creates a rate-limiting barrier for diffusion of the hormone across the skin and allows the dermally administered hormone to be systemically released over an extended period of time, usually up to about 24 hours. In one embodiment of the invention, the average tₘₐₓ (i.e., the time until the maximum concentration of hormone in the blood is achieved) is between about 5 to about 20 hours. In a preferred embodiment the tₘₐₓ is between about 12 and about 20 hours. In another embodiment of the invention, the average tₘₐₓ is between about 2 to about 3 hours.

The hormone delivery system of the present invention is designed to deliver a specific amount of the hormone composition to the skin of an animal. Preferably the animal is a human, but the invention also extends to the treatment of non-human animals.

Diseases or conditions that may be treated by using the drug delivery system and methods of the present invention include, but are not limited to, male hormone therapy in testosterone deficient hypogonadal men, female hormone therapy for postmenopausal women, androgen therapy for females lacking libido, male contraception and female contraception. In preferred embodiments, the hormone is an estrogen, or a derivative thereof, and the intended therapy is for the treatment of the symptoms of menopause in postmenopausal women, e.g., hot flashes, osteoporosis, etc.

In addition to providing improved percutaneous absorption efficiency, the hormone delivery system of the invention may also provide lower irritancy than some other more occlusive delivery systems such as transdermal patches, because the composition is non-occlusive to the skin.

### Hormones

The hormone delivery system of the present invention may deliver one or more hormones and/or one or more hormone derivatives.

Hormones that may be used in the drug delivery system of the present invention include any systemically active hormones which are compatible with the dermal penetration enhancers of the present invention and which can be delivered through the skin with the assistance of the dermal penetration enhancer to achieve a desired effect. Suitable hormones include testosterone, estradiol, ethinyloestradiol, progesterone, norethisterone acetate, gestodene, estriol, estrone, mestranol, stilboestrol, dienoestrol, epioestriol, estropipate, zeranol, allyloestrenol, dydrogesterone, lynoestrenol, nestorone, norgestrel, norethyndrel, norethisterone, gestodene, levonorgestrel, medroxyprogesterone, megestrol and MENT (7-methyl-19-testosterone). In a preferred embodiment, the hormone is an estrogen. In a particularly preferred embodiment the hormone is estradiol.

In one embodiment of the invention, about 1 mg to about 2.5 mg of the hormone is delivered to the skin per spray application. One or more applications of the spray to the skin may be used to achieve a particular therapeutic dose of the hormone.

In one embodiment of the invention, the spray hormone composition results in average serum hormone levels of about 5 pg/ml to about 125 pg/ml of the hormone. In a preferred embodiment, the average serum hormone levels are about 15 pg/ml to about 40 pg/ml, and most preferably the average serum hormone levels are about 20 pg/ml to about 30 pg/ml.

For certain indications or for certain patients, a lower serum concentration of the hormone may be appropriate. Thus, in another embodiment of the invention, the spray hormone composition results in an average serum hormone levels of about 5 pg/ml to about 30 pg/ml, and preferably from about 5 pg/ml to about 20 pg/ml.

In one embodiment of the invention, the dose of the hormone delivered to the skin is about 1 mg per day to about 10 mg per day. In a preferred embodiment, the dose of the hormone delivered to the skin is about 1.5 mg per day to about 5 mg per day.

### Penetration Enhancers

A dermal penetration enhancer is an agent that facilitates the passage of an active agent through the skin. The group of dermal penetration enhancing compounds of the present invention is particularly suitable for non-occlusive transdermal delivery of hormones through the skin of an animal. The dermal penetration enhancing compounds are of low toxicity to the skin and thus are safe at high concentration, are non-irritating, and are excellent promoters of percutaneous absorption. In certain preferred embodiments, the dermal penetration enhancer is selected from the FDA's list of compounds that are generally recognized as safe (the “GRAS list”).

The dermal penetration enhancers of the present invention promote the absorption of hormones through the skin while avoiding the significant pharmacological disadvantages and toxicities of prior art enhancers. Additionally, the dermal penetration enhancers exhibit appreciable penetration into the outer layers of the skin, namely the stratum corneum which has previously presented a formidable barrier to percutaneous drug absorption.

The dermal penetration enhancers of the present invention penetrate the skin and enhance transdermal flux of the hormone.

In vivo flux rate is the average hourly absorption of the drug (into the circulatory bloodstream) per unit area of application (usually cm²). Depending on the duration of the application, the amount of drug that enters circulation over the application period (e.g., daily) is measured, directly or indirectly, and this quantitative measure is divided by the time period (hours) and surface area of application (cm²).

Estimation of quantitative drug absorption over the application period can either be calculated (estimated) by pharmacokinetic mathematics or measured by subtracting the residual drug present at/in the site of application from the quantity applied. Different analytical methods can be employed to measure the “residual” drug. These include extraction by tape stripping the strateum corneum and analysis or measurement of an indirect “marker” by means such as fluorescence or radio-assay.

The hormone, when applied to the skin via the spray composition of the present invention, preferably has an in vivo flux rate of about 0.03 to about 0.3 μg/cm² per hour.

In vitro flux rate can be measured by Franz-type cell experiments as described in U.S. Pat. No. 6,818,226. In vitro flux rate models may utilize different skin types (i.e., mouse, human, etc.) and different receiver fluids (i.e., saline, Sorensen's buffer, etc.).

The dermal penetration enhancers of the present invention have an in vitro flux rate of about 0.01 to about 0.4 μg/cm² per hour. In a preferred embodiment, the dermal penetration enhancers of the present invention have an in vitro flux rate of about 0.2 to about 0.3 μg/cm² per hour.

The dermal penetration enhancers of the present invention have a high octanol/water partition coefficient, preferably at least about 2, and more preferably at least about 3. Also, it is preferred that the penetration enhancer is not charged, or that the penetration enhancer is in its unionized form. This allows a sufficient amount of the penetration enhancer to be taken up in the stratum corneum for effective uptake and permeation enhancement. Preferably, the penetration enhancer should be effective in enhancing drug permeation, preferably an enhancement factor of at least 2-fold.

In addition, the dermal penetration enhancers of the present invention are miscible with volatile organic solvents, particularly with the volatile organic solvent(s) used in the formulation. Thus, in preferred embodiments the dermal penetration enhancers have δ between about 9 and about 18 (cal^{1/2} cm^{-3/2}), and preferably have 6 between about 10 and about 16. This allows the dermal penetration enhancers to be miscible in the volatile solvent (i.e., δ=13 for ethanol and δ=12 for isopropanol). The high solubility of the dermal penetration enhancer in the volatile solvent prevents phase separation or precipitation of the dermal penetration enhancer during the drying process on the skin.

The dermal penetration enhancer preferably has a molecular weight of less than 500 Daltons for effective permeation into the stratum corneum during the preferred solvent drying time upon application to the skin.

Due to the superior ability to penetrate the skin, the dermal penetration enhancers of the present invention allow for a lower volume of the hormone composition to be applied to the skin and a smaller surface area of skin for the application. In preferred embodiments the total volume of the hormone composition that is applied to the skin is between about 75 and about 100 μl, for a single spray of the composition. Also, in preferred embodiments of the invention, the surface area on the skin to which each spray or mist application is applied is from about 10 cm² to about 30 cm².

Penetration enhancers of the present invention may be selected from the groups including terpenes, terpenoids, essential oils, pyrrolidones, azones, fatty acids and esters, sulfoxides, amides, alcohols, glycols and glycerides, amino acid derivatives, phospholipids, surfactants, cyclodextrin complexes, and other groups.

Penetration enhancers that are terpenes, terpenoids, or essential oils include 1-menthol, eucalyptus oil, peppermint oil, turpentine oil, cineole, 1,8-cineole, eucalyptol, d-limonene, α-pinene, nerolidol, α-bisabolol, terpinol, 3-carene, terpinen-4-ol, carveol, carvone, pulgone, piperitone, menthone, cyclohexene oxide, limonene oxide, pinene oxide, cyclopentene oxide, ascaridole, and 7-oxabicyclo(2-2-1)heptane.

Penetration enhancers that are pyrrolidones and azones include N-methyl-2-pyrrolidone (NMP), 2-pyrrolidone, 1-propyl-3-dodecyl-2-pyrrolidone, 1-butyl-3-dodecyl-2-pyrrolidone, 1-ethyl-2-pyrrolidone, 1-hexyl-2-pyrrolidone, 1-butyl-2-pyrrolidone, 1-octyl-2-pyrrolidone, N-dodecyl-2-pyrrolidone, N-(2-hydroxyethyl)-2-pyrrolidone, 1-dodecylazacycloheptan-2-one (azone), 1-geranylazacycloheptone-2-one, 1-farnesylazacycloheptone-2-one, 1-geranylazacyclopentan-2,5-dione, 1-farnesylazacyclopentan-2-one, N-dodecyl-2-piperidinone, 2-(1-nonyl)-1,3-dioxolane (SEPA), cyclopentadecalactone (CPE-215), 1-[2-(decylthio]ethyl)azacyclopentan-2-one (HIPE-101), 4-decyloxazolid-2-one (Dermac SR-38).

The term azones, as used herein refers to 1-alkylazacycloheptan-2-one, wherein the alkyl group has from 8 to 16 carbon atoms.

Penetration enhancers that are fatty acids and esters include, oleic acid, linoleic acid, capric acid, lauric acid, neodecanoic acid, myristic acid, fatty acid extract of cod liver oil, isopropylmyristate, valeric acid, heptanoic acid, pelargonic acid, isovaleric acid, neopentanoic acid, neoheptanoic acid, neononanoic acid, isostearic acid, myristoleic acid, palmitoleic acid, gondoic acid, erucic acid, a-linolenic acid, arachidonic acid, asclepic acid, petroselinic acid, elaidic acid and esters thereof. Preferred esters include alkyl esters, particularly those having from 6-24 carbon atoms, which may be unbranched or branched, saturated or unsaturated, and which may be cyclic or contain a cycloalkyl portion, and which may be unsubstituted or substituted with one or more substituents selected from lower alkoxy, hydroxyl, oxo, halo, and amino.

In certain preferred embodiments, the penetration enhancer is selected from a compound having the formula Iₐ or I_{b}:

R¹—C(═O)—O—H Iₐ

or

R²—C(═O)—O—R³ I_{b}

wherein:
R¹ is selected from a straight chain, branched, or cyclic-containing alkyl group or substituted alkyl group having 6 to 20 carbons, and a straight chain, branched, or cyclic-containing alkenyl group or substituted alkenyl group having 8 to 20 carbons;
R² is selected from a straight chain, branched, or cyclic-containing alkyl group or substituted alkyl group having 6 to 20 carbons, and a straight chain, branched, or cyclic-containing alkenyl group or substituted alkenyl group having 8 to 20 carbons; and
R³ is selected from lower alkyl, lower alkenyl, a straight chain, branched, or cyclic-containing alkyl group or substituted alkyl group having 4 to 14 carbons, and a straight chain, branched, or cyclic-containing alkenyl group or substituted alkenyl group having 4 to 14 carbons; and
the substituted alkyls or substituted alkenyls have from 1 to 4 substituents selected from hydroxy, halo, oxo, alkoxy, and amino.

When the alkyl or alkenyl groups contain a cyclic portion, the cyclic portion may have from 3-7 carbon atoms in the ring. The cyclic portion may be saturated or may contain a double bond between adjacent carbons. Also, the cyclic portion may contain up to two hetero atoms (i.e., O, S, or N) in place of one of the 2-7 carbon atoms in the ring. The cyclic portion may be unsubstituted, or may be optionally substituted with 1 to 4 substituents selected from lower alkyl, lower alkoxy, hydroxyl, oxo, halo, and amino.

Alkyl and alkoxy groups referred to herein may be either straight chain or branched. The term “lower alkyl” refers to alkyl groups containing from 1 to 5 carbon atoms. The term lower alkoxy refers to the group —O-(lower alkyl). The term “halide” or “halo” means fluoride, chloride, bromide or iodide. The term “amino” refers to —NH₂, —NH(lower alkyl), or —N(lower alkyl)₂.

Penetration enhancers that are amides include dimethylacetamide, N,N-dimethyloctanamide, and N,N-dimethyldecanamide. Preferred amides have the formula II:

R⁴—C(═O)—N(R⁵)(R⁶) II

R¹ is selected from a straight chain, branched, or cyclic-containing alkyl group or substituted alkyl group having 2 to 20 carbons, and a straight chain, branched, or cyclic-containing alkenyl group or substituted alkenyl group having 2 to 20 carbons;
R⁵ is selected from a straight chain, branched, or cyclic-containing alkyl group or substituted alkyl group having 1 to 16 carbons, and a straight chain, branched, or cyclic-containing alkenyl group or substituted alkenyl group having 1 to 16 carbons; and
R⁶ is selected from H, a straight chain, branched, or cyclic-containing alkyl group or substituted alkyl group having 1 to 14 carbons, and a straight chain, branched, or cyclic-containing alkenyl group or substituted alkenyl group having 2 to 14 carbons; and
the substituted alkyls or substituted alkenyls have from 1 to 4 substituents selected from hydroxy, halo, oxo, alkoxy, and amino.

Penetration enhancers that are alcohols, glycols and glycerides include: propylene glycol, octyl alcohol, lauryl alcohol, glycerin tricaprylate (caprylic acid triglyceride), glyceryl monocaprylate, Sefsol 318 (medium-chain glyceride, monoglycerides, polyglycosylated glycerides, Transcutol, poyethylene glycol 400, and polycylcolized glyceride.

Preferred alcohols have from 5-20 carbon atoms, which may be unbranched or branched, saturated or unsaturated, and which may be cyclic or contain a cycloalkyl portion, and which may be unsubstituted or substituted with one or more substituents selected from lower alkoxy, hydroxyl, oxo, halo, and amino.

Particularly preferred alcohols include alkyl alcohols, particularly those having from 5-20 carbon atoms, and which may be unbranched or branched, which may be cyclic or contain a cycloalkyl portion, and which may be unsubstituted or substituted with one or more substituents selected from lower alkoxy, hydroxyl, oxo, halo, and amino.

Penetration enhancers that are sulfoxides include: dimethyl sulfoxide, and decylmethyl sulfoxide. Preferred sulfoxides have the formula:

(C₂-C₁₆ alkyl)-S(—O)—(C₁-C₁₆ alkyl), and particularly preferred sulfoxides have the formula (C₄-C₁₆ alkyl)-S(═O)—(C₁-C₃ alkyl).

Penetration enhancers that are amino acid derivatives include: N-dodecyl-1-amino acid methyl ester, n-pentyl-N-acetyl prolinate, octyl-6-aminohexanoate, decyl-6-aminohexanoate, dodecyl-N,N-dimethylamino isopropionate, and dodecyl-N,N-dimethylamino acetate.

Penetration enhancers that are phospholipids include: phosphatidyl glycerol derivatives, phosphatidyl choline derivates, and phosphatidyl ethanolamine derivatives.

Penetration enhancers that are surfactants include: bile salts, polysorbates, and sodium lauryl sulfate.

Penetration enhancers that are cyclodextrin complexes include: β-cyclodextrin and methyl-β-cyclodextrin.

Other preferred penetration enhancers include: alkyl-2-(N,N-disubstituted amino)-alkanoate ester (NexAct), N-acetylprolineesters, neohesperidinedihydrochalcone, fatty acid esters of lactic acid salts, polyethyleneglycol monoalkyl ethers, crotamiton, levulic acid, sterols and sterol esters, acyl lactylates, oleic acid dimers, neodecanoic acid, dioxolanes, polyoxyethylene cetyl ethers, methyl laurate, glycerol monolaurate, and esters and amides of clofibric acid.

In certain embodiments, particularly preferred penetration enhancers include: butylated hydroxyanisole, 2-phenoxyethanol, thymol, menthol, menthone, cineole, isopropyl myristate, glyceryl monolaurate, glyceryl monostearate, glyceryl monooleate, oleic acid, oleyl alcohol, methyl laurate, sorbitan monooleate, lauryl lactate, and lauryl alcohol.

In certain embodiments of the invention, preferred dermal penetration enhancers include fatty acids and fatty acid esters and derivatives thereof. Preferably, the fatty acid moiety of the fatty acids, fatty acid esters and derivatives thereof does not contain an aromatic group. In other embodiments, the fatty acid portion of the fatty acid ester and the alcohol portion of the ester are selected from linear or branched alkyl groups.

### Volatile Solvents

It is most desirable that, after application of the hormone composition to the skin, the volatile component of the composition evaporates and the area of skin to which the hormone composition was applied becomes touch-dry. Preferably said area of skin becomes touch-dry within 10 minutes, more preferably within 3 minutes, more preferably within 2 minutes, and most preferably within 1 minute. In one embodiment the volatile solvent evaporates faster that ethanol.

Preferred volatile solvents of the present invention include safe skin-tolerant solvents such as ethanol, isopropanol and ether. An aerosol propellant, such as dimethyl ether, may constitute a volatile liquid for the purpose of the present invention.

### Formulation

In one preferred formulation of the invention the hormone composition comprises on a weight basis from about 0.1 to about 10% of the hormone, from about 0.1 to about 12% of the at least one dermal penetration enhancer and from about 78 to 99.8% of solvent.

In another preferred formulation of the invention the hormone composition comprises on a weight basis from about 1% to about 5% of the hormone, from about 0.01% to about 10% of the at least one dermal penetration enhancer, from about 60 to 99% of a solvent or cosolvent, and from about 0% to about 30% of other adjuvants.

In another preferred formulation of the invention the hormone composition comprises, on a weight basis, from about 1 to about 3% of a hormone, from about 1 to about 12% of at least one dermal penetration enhancer, from about 45% to about 90% ethanol, isopropanol or mixture thereof, and about 5 to about 45% water or other co-solvant.

In one preferred formulation of the invention the hormone composition comprises on a weight basis from about 0.1% to about 5% of a stabilizing agent.

While it is preferred that the hormone and penetration enhancer be delivered by simultaneous administration, the penetration enhancer may be applied before or after the application of the hormone, if desired.

Preferably the hormone composition is not supersaturated with respect to the hormone. As the volatile liquid of the hormone composition evaporates, the resulting composition is rapidly driven into the dermal surface. It is possible that as the volatile liquid evaporates, the dermal penetration enhancer becomes supersaturated with respect to the hormone. However, it is preferred that any supersaturation does not occur before transport of the resulting composition across the epidermal surface has occurred.

In the hormone delivery systems according to the present invention a pharmaceutical compounding agent, co-solvent, surfactant, emulsifier, antioxidant, preservative, stabilizer, diluent or a mixture of two or more of said components may be incorporated in these systems as is appropriate. The amount and type of components used should be compatible with the dermal penetration enhancers of this invention as well as with the hormone. A co-solvent or other standard adjuvant, such as a surfactant, may be used if needed to maintain the hormone in solution or suspension at the desired concentration.

The pharmaceutical compounding agents can include paraffin oils, esters such as isopropyl myristate, ethanol, silicone oils and vegetable oils. These are preferably used in the range 1 to 50%. Surfactants such as ethoxylated fatty alcohols, glycerol mono stearate, phosphate esters, and other commonly used emulsifiers and surfactants preferably in the range of 0.1 to 10% may be used, as may be preservatives such as hydroxybenzoate esters for preservation of the compound preferably in amounts of 0.01% to 0.5%. Typical co-solvents and adjuvants may be isopropyl alcohol, acetone, dimethyl ether and glycol ethers such as diethylene glycol mono ethyl ether. These may be used in amounts of 1 to 50%.

Optionally the hormone delivery system may contain pharmaceutical compounding agents, such as one or more thickening agents such as cellulosic thickening agents, ethylcellulose, hydroxypropyl cellulose, hydroxypropyl methylcellulose, povidone, polyacrylic acids such as carbopol, Sepigel™. (polyacrylamide/isoparaffin/laureth-7), the Gantrez™. series of polymethyl vinyl ether/maleic anhydride copolymers such as the butyl ester of PVM/MA copolymer Gantrez™. A-425, and any thickening agent known in the art that has good compatibility with the volatile liquid and enhancers of the present invention.

Because of the effect of the penetration enhancer of the invention, the dosage of the hormone may often be less than that conventionally used. It is proposed that, a dosage near the lower end of the useful range of the particular hormone may be employed initially and increased as indicated from the observed response if necessary.

The concentration of hormone used in the drug delivery system will depend on its properties and may be equivalent to that normally utilized for the particular hormone in conventional formulations. Both the amount of hormone and the amount of penetration enhancer will be influenced by the type of effect desired.

Where it is desired to achieve higher systemic concentration of a hormone, proportionately higher concentrations of the enhancer of the invention may be required in the transdermal drug delivery system of the present invention, and the amount of hormone included in the composition should be sufficient to provide the blood level desired.

The concentration of penetration enhancer may be in the range from about 10 to about 1,000 weight percent of penetration enhancer based upon the weight of hormone. In a preferred embodiment, the concentration of the penetration enhancer is from about 50 to about 500 weight percent of the penetration enhancer based upon the weight of the hormone. The ratio of penetration enhancer to hormone may vary considerably and will be governed as much as anything, by the pharmacological results that are required to be achieved. In principle, it is desirable that as little penetration enhancer as possible is used. On the other hand, for some hormones, it may be that the upper range of 500% by weight will be required. It is preferred that the penetration enhancer and hormone are in approximately equal proportions.

A particular advantage of the hormone delivery system of the present invention is that patient compliance is improved as the system does not occlude the skin. As a result, local irritation and allergic sensitization problems arising from prolonged exposure of the skin to both the delivery system of occlusive transdermal patch devices and the adhesive used to affix these patches to the skin are reduced.

### Device

The hormone delivery system of the present invention includes a device capable of delivering to the skin a metered dose of the hormone composition in the form of a mist or spray.

It is one of the objects of the present invention to provide a hormone composition dispensing device having means for preventing, or minimizing, unintentional loss of the hormone composition. It is a further object of the present invention to provide a hormone composition dispensing device that is able to dispense an accurately metered quantity of a hormone composition during normal use. It is yet another object of the present invention to provide a hormone composition dispensing device having means to enable a full charge of a substance to be available for discharge preparatory to normal use of the device. Still another object of the present invention is to provide a dispensing device that is particularly suitable for use in transdermal application of hormone compositions.

A hand-held dispensing device for dispensing and applying compositions to the skin is described in U.S. Pat. No. 6,978,945, which is incorporated herein by reference in its entirety. The device includes a hollow body, a capsule mounted within the hollow body and including the drug composition therein, a nozzle having an outlet passage mounted within the hollow body in communication with the capsule, actuator means operable to cause a metered quantity of the drug composition to be dispensed from the capsule through the outlet passage of the nozzle, a shroud defining an exit space from the hollow body for receiving the drug composition emerging from the outlet passage, and closure means including a cap detachably mountable on the shroud to thereby selectively open or close the nozzle and thereby control escape of the drug composition from the capsule. In a preferred embodiment, the outer passage includes an exit end, and the closure means includes a closure member that is operative to close the outlet passage by engaging against a surface surrounding the exit end of the outlet passage. Preferably, the device includes force inducing means operative to apply a closing force to the closure member when the closure member is engaged with the surface surrounding the exit end of the outlet passage.

In accordance with one embodiment of the dispensing device of the present invention, the shroud includes a first end proximate to the nozzle and a second end distal from the nozzle, the second end including an open mouth, whereby the shroud confines the lateral spread of the substance during its passage from the nozzle to the open mouth. Preferably, the shroud is substantially conical and includes a progressively increasing size from the first end to the second end. In another embodiment, the cap is detachably mountable to the second end of the shroud and extends across and closes the open mouth when the closure means is operated to close the nozzle and does not extend across the open mouth when the closure means is operated to open the nozzle. In a preferred embodiment, the outlet passage includes an exit end, and the closure means includes an elongated closure member having a first end connected to the cap and a second end which is engageable with a surface associated with the exit end of the outlet passage to thereby close the nozzle. Preferably, the device includes force inducing means operative to apply a closing force to the elongated closure member when the elongated closure member is engaged with the surface associated with the exit end of the outlet passage. In a preferred embodiment, the cap includes a flexible end wall and the force inducing means includes the flexible end wall of the cap, the first end of the elongated closure member is connected to a mid region of the flexible end wall, and the flexible end wall is resiliently distorted so as to place the elongated closure member under compression when the cap is attached to the outer end of the shroud and the elongated closure member engages with the surface associated with the exit end of the outlet passage. Preferably, the cap is releasable snap-engageable with the second end of the shroud.

In accordance with one embodiment of the dispensing device of the present invention, the distance between the nozzle and the second end of the shroud is predetermined, whereby the shroud may be used to regulate the distance between the nozzle and a target area onto which the substance is to be deposited. Preferably, the size of the open mouth is substantially equal to the size of the target area.

In accordance with another embodiment of the dispensing device of the present invention, the device includes guide means disposed adjacent the nozzle to receive at least part of the closure member and guide the closure member into correct engagement with the surface surrounding the exit end of the outlet passage when the closure means is mounted on the shroud to close the nozzle.

In accordance with another embodiment of the dispensing device of the present invention, the closure member includes flexible sealing means for engaging against the surface surrounding the exit end of the outlet passage. In a preferred embodiment, the flexible sealing means includes a flexible circular sealing lip that surrounds the exit end of the outlet passage when engaged with the surface surrounding the exit end of the outlet passage. Preferably, the closure member defines a cavity communicating with the exit end of the outlet passage when the flexible sealing means and the surface surrounding the exit end of the outlet passage are in engagement.

In accordance with the present invention, a hand-held device for dispensing and applying a substance to the skin of a host includes a hollow body, a capsule mounted with the hollow body, a container for the substance forming part of the capsule and including the substance therein, a nozzle mounted within the hollow body, a pump operable to cause a metered quantity of the substance to be dispensed from the capsule through the nozzle in the form of a spray, an actuator mounted on the hollow body and being operable to cause operating of the pump, and absorption means locatable at a first location in alignment with the nozzle and at a second location out of alignment with the nozzle, whereby when the absorption means is at the first location it can absorb a pre-use quantity of the substance dispensed through the nozzle during at least a first of a series of operations of the actuator means, and when the absorption means is at the second location it can enable a full charge of the substance to be dispensed through the nozzle during a subsequent one of the series of operations of the actuator means. In a preferred embodiment, the device includes a cap member removably connectable to the dispensing device, the absorption means including an absorbent pad attached to the cap member, whereby when the cap member is connected to the dispensing device the absorbent pad is positioned in the path of the substance being dispensed through the nozzle. Preferably, the device includes a shroud defining an exit space for receiving the substance emerging from the nozzle, the shroud including a first end proximate to the nozzle and a second end distal from the nozzle, the second end including an open mouth, and the cap being removably connectable to the second end of the shroud.

In accordance with another embodiment of the dispensing device of the present invention, the device includes absorption means locatable at a first location in alignment with the nozzle and a second location out of alignment with the nozzle, whereby when the absorption means is in the first location it can absorb a pre-use quantity of the substance dispensed through the nozzle during at least the first of a series of operations of the actuator means, and when the absorption means is at the second location it can enable a full charge of the substance to be dispensed through the nozzle during a subsequent one of the series of operations of the actuator means. In a preferred embodiment, the absorption means includes an absorbent pad attached to the cap, whereby the absorbent pad is positioned in the path of the substance being dispensed through the nozzle when the cap is mounted on the shroud. More preferably, the absorbent pad is releasably attached to the cap.

In accordance with another embodiment of the dispensing device of the present invention, the shroud provides a non-vented wall around the exit space.

In accordance with another embodiment of the dispensing device of the present invention, the capsule is removably mounted within the hollow body. Preferably, the hollow body comprises a first part and a second part, each of the first and second parts defining a respective portion of a chamber within which the capsule is mounted.

In accordance with another embodiment of the dispensing device of the present invention, the actuator means includes a pump connected to the nozzle so as to be operable to withdraw the substance from the capsule and expel the withdrawn substance through the nozzle. In a preferred embodiment, the actuator means includes a button movably mounted on the hollow body and operable to cause operation of the pump.

In accordance with another embodiment of the dispensing device of the present invention, the actuator means includes a valve, the substance is pressurized, and the valve is selectively operable to permit dispersion of the substance in aerosol form from the nozzle. Preferably, the actuator means includes a button movably mounted on the hollow body and operable on a selective basis to open the valve.

In accordance with another embodiment of the dispensing device of the present invention, the surface surrounding the exit end of the outlet passage is a frusto-conical surface, and the closure member includes a substantially complementary surface engageable with the frusto-conical surface.

In accordance with another embodiment of the dispensing device of the present invention, the hollow body is adapted to be grasped by the hand of a user and includes a major axis that extends transverse to the fingers of the user where grasped, and the nozzle is disposed to dispense the substance in a lateral direction relative to the major axis. Preferably, the shroud provides a non-vented wall around the exit space.

In accordance with another embodiment of the dispensing device of the present invention, the pump is connected to the nozzle, whereby the pump is operable to withdraw the substance from the container and expel the withdrawn substance through the nozzle.

According to one aspect of the present invention there is provided a hand-held device for dispensing a substance and applying that substance to the skin of a host, the device including a hollow body, a substance capsule mounted within the body, a container for the substance forming part of the capsule, a spray nozzle having an outlet passage, actuator means connected to the interior of the container and being operable to cause a metered quantity of the substance to be dispensed through the outlet passage of the nozzle, a shroud defining a space for receiving the substance emerging from the outlet passage, and closure means including a cap detachably located on the shroud selectively operable to close or open the nozzle and thereby control escape of the substance from the capsule.

A transdermal spray applicator device according to the present invention may be charged with a substance in the form of a single-phase volatile/non-volatile liquid provided within a standard plastic and/or glass container (depending on the characteristics of the active ingredient). Manufacture of the device in one of its preferred forms is relatively straight-forward, amenable to simple scale-up, and uses “off-the-shelf” components for the primary pharmaceutical packaging. That contrasts with the complexities involved in manufacturing occlusive patch devices as described above.

In a preferred form of the device, the nozzle outlet communicates with a space defined within a shroud of suitable configuration and size. Such a shroud is particularly useful in circumstances involving transdermal application of a substance, because it can assist in ensuring that the dispensed substance is confined to the intended target area. It is preferred that the shroud provides a complete non-vented enclosure over the target area so as to reduce the risk of spray drift and consequent loss of some of the substance being dispensed. Furthermore, the shroud can function as a distance regulating device. That is, when the open mouth of the shroud engages a surface surrounding the intended target area, the distance between the target area and the outlet of the spray nozzle is preferably substantially equal to the ideal distance over which the substance should be sprayed on to the target area.

Reference to “non-vented” in the previous paragraph and other parts of this specification is not to be understood as demanding complete absence of exposure to the atmosphere. The preferred shroud is “non-vented” in the sense that it does not have openings deliberately formed through the side wall (e.g. as in the device of U.S. Pat. No. 6,113,008), or through the outer edge intended to engage against the surface surrounding the target area.

The nozzle closure means may be formed by or provided on a cap that is adapted to close the open mouth of the shroud when the device is not in use. By way of example, the closure means may include an elongated stem that extends outwards from a wall of the cap. The arrangement is such that the end of the stem remote from the cap wall is engageable within and/or around the nozzle outlet when the cap is in place on the shroud. It is preferred that the cap wall is flexible and is subjected to resilient distortion when the cap is attached to the shroud. The stress thereby generated within the cap wall tends to push the closure stem against the nozzle outlet and thereby maintain a suitable closing force between the stem and the outlet.

According to another aspect of the present invention, there is provided a substance dispensing device having means for enabling a full charge of a substance to be available within the delivery system of the device for discharge when the device is operated in a normal manner. In that regard, a “full charge” is to be understood as comprising a quantity of the substance substantially equal to the melted amount intended to be discharged form the device when operated in a correct manner.

A condition called “loss of prime” can occur in the delivery system while the device is not in use. One cause of such loss of prime is evaporation, particularly when volatile substances are being used. As a result, air occupies a space within the delivery system that was intended to be occupied by the substance. Thus, when the device is thereafter operated to discharge the substance, the quantity discharged will be less than the intended metered quantity.

In order to enable a full charge of the substance to be available for discharge, a device in accordance with a second aspect of the present invention may be provided with means for absorbing a pre-use quantity of the substance. Prior to normal use of the device, the absorption means is placed in alignment with the nozzle outlet, and the device is operated to cause a pre-use quantity of the substance to be directed onto the absorption means. The delivery system of the device is thereby primed in the sense that it thereafter contains a full charge of the substance. Removal of the absorbing means from exposure to the nozzle outlet enables the device to be operated to discharge the intended metered quantity of the substance.

The absorption means referred to above can be substituted for the closure means of the first described aspect of the invention. That is, it may not be necessary to provide a device with both the nozzle closure means and the priming means as described above. Each has the ability to control loss of prime so as to thereby improve the efficiency and effectiveness of substance dispensing devices, and is therefore useful in its own right. On the other hand, a device could incorporate both the closure means and the priming means if desired.

A device according to either aspect of the present invention as described above could be disposable or rechargeable. That is, in one form, the entire device may be discarded when the contents of the capsule are exhausted, whereas in another form the capsule may be removably mounted in the hollow body of the device so as to enable removal and replacement by a fully charged capsule.

Also, in a device according to either of the two described aspects of the present invention, the form of the actuator means may differ according to whether the substance is dispensed by means of a manually operable pump, or by an aerosol-type process. In the former case, the pump may form part of the actuator means. In the latter case the actuator means may include a valve that is operable to connect the nozzle to the pressurized contents of the substance container.

The terms “percutaneous” and “transdermal” are used herein in the broadest sense to refer to being able to pass through unbroken skin.

The term “non-occlusive” is used herein in its broadest sense to refer to not trapping or closing the skin to the atmosphere by means of a patch device, fixed reservoir, application chamber, tape, bandage, sticking plaster, or the like which remains on the skin at the site of application for a prolonged length of time.

The term “stratum corneum” is used herein in its broadest sense to refer to the outer layer of the skin, which is comprised of (approximately 15) layers of terminally differentiated keratinocytes made primarily of the proteinaceous material keratin arranged in a ‘brick and mortar’ fashion with the mortar being comprised of a lipid matrix made primarily from cholesterol, ceramides and long chain fatty acids. The stratum corneum creates the rate-limiting barrier for diffusion of the active agent across the skin.

The term “skin-depot” is used herein in its broadest sense to refer to a reservoir or deposit of active agent and dermal penetration enhancer within the stratum corneum, whether it be intra-cellular (within keratinocytes) or inter-cellular.

### EXAMPLES

The formulations provided below are intended to represent possible hormone spray compositions of the present invention. It is understood that the materials and amounts do not necessarily limit the scope of the invention.

### Example 1

A formulation consisting of:

| | | |
|---|---|---|
| Estradiol | 10-20 | mg/mL |
| Isopropyl Myristate | 8.0% | w/v |
| Ethyl Alcohol | 42% | v/v |
| Ethyl Ether | 50% | v/v |

The formulation is sprayed from a metered dose sprayer device onto the skin using a fabricated spacer/shroud, the application covering 20 cm² to 60 cm² surface area of the skin. The liquid deposited on the skin evaporates within about 2 minutes leaving an almost imperceptible deposit on the skin. Resultant blood levels of estradiol will be achieved within about 2 to about 4 hours which produce a therapeutic effect of the drug lasting at least about 24 hours after administration. The target blood levels are between about 20 pg/ml to about 30 pg/ml.

### Example 2

A formulation consisting of:

| | |
|---|---|
| Testosterone | 2% w/v |
| Laurocapram | 5% w/v |
| Ethyl Alcohol | 93% v/v |

The formulation is sprayed from a metered dose sprayer device onto the skin using a fabricated spacer/shroud, the application covering about 20 cm² to about 60 cm² surface area of the skin. The liquid deposited on the skin evaporates within 2 minutes leaving an almost imperceptible deposit on the skin. Resultant blood levels of testosterone will be achieved within about 2 to about 4 hours which produce a therapeutic effect of the drug lasting at least 24 hours after administration. The target blood levels are between about 0.1 ng/mL and about 0.5 ng/mL.

1. A transdermal estrogen spray composition, comprising:
a therapeutically effective amount of an estrogen;
a penetration enhancer; and
a volatile solvent,
wherein the penetration enhancer is selected from terpenes, terpenoids, essential oils, pyrrolidones, azones, sulfoxides, amides, alcohols, glycols, glycerides, amino acid derivatives, phospholipids, surfactants, and fatty acids and esters thereof having the formula Iₐ or I_{b}:
R¹—C(═O)—O—H Iₐ
or
R²—C(═O)—O—R³ I_{b}
wherein:
R¹ is selected from a straight chain, branched, or cyclic-containing alkyl group or substituted alkyl group having 8 to 20 carbons, and a straight chain, branched, or cyclic-containing alkenyl group or substituted alkenyl group having 8 to 20 carbons;
R² is selected from a straight chain, branched, or cyclic-containing alkyl group or substituted alkyl group having 8 to 20 carbons, and a straight chain, branched, or cyclic-containing alkenyl group or substituted alkenyl group having 8 to 20 carbons; and
R³ is selected from lower alkyl, lower alkenyl, a straight chain, branched, or cyclic-containing alkyl group or substituted alkyl group having 6 to 14 carbons, and a straight chain, branched, or cyclic-containing alkenyl group or substituted alkenyl group having 6 to 14 carbons; and
the substituted alkyls or substituted alkenyls have from 1 to 4 substituents selected from hydroxy, halo, oxo, alkoxy, and amino; and
wherein, when applied to the skin, the composition provides an in vivo transdermal flux of estrogen of about 0.03 to about 0.3 μg/cm² per hour.

2. The transdermal estrogen spray of claim 1, wherein the estrogen is estradiol.

3. The transdermal estrogen spray of claim 1, wherein the penetration enhancer is selected from the groups consisting of terpenes, terpenoids, essential oils, pyrrolidones, azones, fatty acids, fatty acid esters, sulfoxides, amides, alcohols, glycols and glycerides, amino acid derivatives, phospholipids, and surfactants.

4. The transdermal estrogen spray of claim 3, wherein the penetration enhancer is a fatty acid or a fatty acid ester.

5. The transdermal estrogen spray of claim 4, wherein the penetration enhancer is selected from a compound having the formula Iₐ or I_{b}:
R¹—C(═O)—O—H Iₐ
or
R²—C(═O)—O—R³ I_{b}
wherein:
R¹ is selected from a straight chain, branched, or cyclic-containing alkyl group or substituted alkyl group having 8 to 20 carbons, and a straight chain, branched, or cyclic-containing alkenyl group or substituted alkenyl group having 8 to 20 carbons;
R² is selected from a straight chain, branched, or cyclic-containing alkyl group or substituted alkyl group having 8 to 20 carbons, and a straight chain, branched, or cyclic-containing alkenyl group or substituted alkenyl group having 8 to 20 carbons; and
R³ is selected from lower alkyl, lower alkenyl, a straight chain, branched, or cyclic-containing alkyl group or substituted alkyl group having 6 to 14 carbons, and a straight chain, branched, or cyclic-containing alkenyl group or substituted alkenyl group having 6 to 14 carbons; and
the substituted alkyls or substituted alkenyls have from 1 to 4 substituents selected from hydroxy, halo, oxo, alkoxy, and amino.

6. The transdermal estrogen spray of claim 3, wherein the penetration enhancer is selected from the group consisting of butylated hydroxyanisole, 2-phenoxyethanol, thymol, menthol, menthone, cineole, isopropyl myristate, glyceryl monolaurate, glyceryl monostearate, glyceryl monooleate, oleic acid, oleyl alcohol, methyl laurate, sorbitan monooleate, lauryl lactate, and lauryl alcohol.

7. The transdermal estrogen spray of claim 1, wherein the solvent is selected from ethanol and isopropanol.
